(19) United States
(12) Patent Application Publication
(10) Pub. No.: US 2026/0345918 A1
(43) Pub. Date: Nov. 5, 2026

(54) INJECTOR DISASSEMBLY TOOL

(71) Applicant: Hemingway Designs LLC, Oregon City, OR (US)

(72) Inventors: Brian W. Brandell, Portland, OR (US); Colt Kincade Stuart, Lake Oswego, OR (US)

(21) Appl. No.: 19/119,170

(22) PCT Filed: Oct. 8, 2023
(86) PCT No.: PCT/US2023/034711

§ 371 Date: Apr. 8, 2025

Related U.S. Application Data
(60) Provisional application No. 63/414,901, filed on Oct. 10, 2022.

(51) Int. Cl. A61M 5/32; B23P 19/04

(52) U.S. Cl. CPC A61M 5/3205; B23P 19/04; A61M 2207/10; A61M 2209/00

(57) ABSTRACT

Multiple example embodiments and methos of use of a device for controlled disassembly of personal disposable injector pens and related injectors is disclosed. Prefilled disposable injection pens that have reached end-of-use, expiration, or are otherwise no longer fit or desired for use cannot be recycled because constituent components are handled differently and the devices are not designed to be disassembled. The injector disassembly device mechanisms enable separation or division of the injection pen housing without breaking or otherwise damaging the drug cartridge so the drug cartridge can be removed from the cartridge holder. Further the mechanism performs the separation of the injection pen housing in a controlled manner so parting of the cartridge housing does not create uncontrolled fragmentation and resulting sharp surfaces which may present a hazard to the user. The parting mechanism accommodates the shape and form factor of at least one commercially offered injection pen.

### BACKGROUND OF THE INVENTION

### Technical Field

The disclosures herein relate to devices and methods for disassembly of disposable injectors. Specifically, the disclosed example embodiments relate to devices and methods for at least partially disassembling a disposable medicament pen injector to allow for recycling of the injector's component materials and other sustainable options for disposal.

### State of the Art

The preferred therapeutic treatment for many chronic diseases today includes daily outpatient injection of medications by patients. For example, persons with diabetes represent a large and growing population that rely on self-injection for treatment. Type 1 and type 2 diabetics both use multiple daily injections of both long-acting and short-acting insulin formulations. The International Diabetes Federation estimates that with the rapid growth of diabetes worldwide, 100 million people with diabetes will be using daily insulin by 2030. In the US today there are an estimated 10 million insulin users.

The growing need for devices used for self-injection parallels the increasing incidence of diabetes and other chronic diseases. To meet this need, manufacturers have developed disposable, prefilled devices commonly known as “pen injectors” for patient use. As the name suggests, a pen injector has a typically elongated, tubular shape, like a pen for writing. The first disposable prefilled pen injector was introduced to the market by Novo Nordisk in 1989. Over the last 34 years, pen injector popularity has grown substantially. For example, persons with diabetes prefer the use of insulin pens over traditional vials and syringes because of the convenience, ease of use, accuracy, discretion, and small needle size. In Europe, approximately 95% of diabetics requiring daily insulin use injector pens. In the United States, the pen injector utilization rate among diabetics is currently about 65% but is growing rapidly as insurance reimbursement has become more universal. The use of pen injectors in the rest of the world is also expanding dramatically.

The popularity of insulin pen injectors has led to the use of prefilled disposable pen injectors to deliver medication for other drugs. Many Type 2 diabetics are using disposable pens for delivery of glucagon-like peptide-1 (GLP-1) receptor agonists. Patients with other conditions, including rheumatoid arthritis and Crohn's disease, are finding therapeutic remedies through periodic self-injections of biologics. Although many insulin pen injectors can be used over multiple doses, pen injectors for other therapeutics are configured as single-dose devices to deliver a single fixed dose and then be discarded. Regardless of single dose or multidose, disposable prefilled pen injectors are currently all designed to be discarded when depleted.

By design, pen injectors are not meant to be disassembled or otherwise modified or tampered with. As a result, prefilled disposable pen injectors cannot be recycled. Pen injectors contain multiple material types that are not readily separable. In addition, a very small amount of residual medicament and potential biological waste may remain in the cartridge even though the injector has become functionally depleted. Thus, approximately 2 billion disposable prefilled pen injectors are disposed of in landfills annually. Pen injectors represent a significant contribution to the global problem of single-use plastics entering our environment.

In response to growing concerns about the environmental impact of single-use plastic, pen injector manufacturers may choose to employ more sustainable, reusable, or recyclable non-plastic materials to form various components of an injector. For example, injector bodies and cartridge holders could be made from metal such as aluminum, or from wood, bamboo, bagasse, or composites. However, glass will likely continue to be used for the manufacture of the cartridge containing the medicament. Glass has a long history in the pharmaceutical industry and with government regulatory agencies when used to form closed-container systems. Even if manufacturers adopt alternative materials for their pen injectors, it is unlikely that improved designs that can be easily disassembled for recycling by users will be advanced.

It would therefore be desirable to provide an apparatus that allows a user to safely and conveniently disassemble a disposable prefilled pen injector. It would also be desirable if the use of the apparatus did not pose significant risk of breaking or damaging the internal glass cartridge. Disassembly would allow the constituent components of the pen injector to be separated for recycling or other modes of disposal. For example, separating the glass cartridge from the plastic components would facilitate recycling of each. Further, it would be desirable if the apparatus could accommodate pen injectors having different lengths and cross-sectional profiles present in commonly used injectors. Further, it would be desirable if the apparatus was able to disassemble disposable prefilled pen injectors having bodies, housings, and various internal components made from more sustainable, reusable, or recyclable materials.

For at least the foregoing issues, the lack of convenient, safe, and easy-to-use disassembly device for pen injectors substantially prevents recycling of the component materials forming a pen injector, contributes to environmental levels of single-use plastics, and is counter to large-scale environmentally sustainable manufacturing practices necessary to meet the rapidly growing global demand for pen injectors.

Consequently, there is a need for a pen injector disassembly tool to address the aforementioned deficiencies.

### BRIEF SUMMARY

Disclosed herein are embodiments of a pen injector disassembly tool.

In some embodiments, the injector disassembly tool comprises a holder section having a fixing means configured to couple to a disposable injector; a cutter section, wherein a drive section mechanically coupled to the cutter mechanism; and a coupler section; wherein the holder section, the cutter section, the drive section, and the coupler section are sequentially coupled end-to-end along a common axis.

In some embodiments, the fixing means is a female threaded fixture. In some embodiments, the holder section, the cutter section, the drive section, and the coupler section are reversibly coupled through an interaction of interlocking surface features joining a housing of each section. In some embodiments, the cutter section comprises a cutting wheel. In some emobdiments, the cutter section comprises a heated wire. In some embodiments, the cutter section comprises a non-rotating cutting blade.

Disclosed is method of use for an injector disassembly tool comprising an inserting step; a coupling step; a rotating step; a cutting step; a releasing step; and a decoupling step.

In some embodiments, the inserting step comprises threading a pen injector into a threaded fixture on a base of an injector disassembly tool. In some embodiments, the coupling step comprises stabilizing the pen injector with a live center mechanism. In some embodiments, the rotating step comprises manually rotating a handle to engage a clutch mechanism mechanically coupled to a cutter wheel.

Disclosed is a device for the disassembly of prefilled disposable injection pens that have reached end-of-use, expired, or are otherwise no longer fit for use. Example embodiments of the disassembly device include a housing, a fixing mechanism, and a parting mechanism. The fixing mechanism acts to hold and support the injection pen in the desired orientation relative to the parting mechanism during disassembly. The fixing mechanism may provide a user-actuated feature for translation of the injection pen along an axis BB, as shown in FIG. 3, or rotation around axis BB to facilitate the cutting and parting mechanisms of the disassembly device. The fixing mechanism accommodates the shape and overall form of at least one commercially offered injection pen. Preferably, the fixing mechanism accommodates the shape and form factor of a large portion of the commercially available injection pens. The fixing mechanism may interact with one or more features of injection pens for fixing, such as the distal threads or twist lock mechanism on the cartridge holder, for example.

The parting mechanism enables the division and separation of the injection pen housing without breaking or otherwise damaging the drug cartridge such that the drug cartridge can be accessed and removed from the cartridge holder. Also, the parting mechanism performs the separation of the injection pen housing in a controlled and consistent manner such that the parting of the housing does not result in uncontrolled fragmentation or otherwise create sharp surfaces which may injure the user. This cutting and separation is accomplished in various ways, according to the embodiment of the injector disassembly tool. For example, in some embodiments, the parting mechanism operates by fully dividing the housing or a housing component into two or more parts. In some embodiments, the parting mechanism includes a user-actuated feature for translation of the parting mechanism along the injection pen's axis AA or rotation around axis AA to facilitate cutting and parting. In some embodiments, the parting mechanism operates by partially dividing the housing or a housing component into two or more parts in such a way that the divided parts can be pivoted or twisted with respect to each other sufficient to remove the medicament cartridge. In some embodiments, the parting mechanism operates by structurally weakening the housing or housing component in such a way that the regions on either side of weakened region can be pivoted or twisted with respect to each other sufficient so that the medicament cartridge can be removed. In some embodiments, the parting mechanism operates by structurally weakening the interface between the cartridge holder and pen body such that the cartridge holder and pen body can be adequately separated for removal of the medicament cartridge.

The parting mechanism is configured to receive the shape and certain surface features of at least one commercially offered injection pen. In some embodiments, the injector disassembly tool accommodates a large portion of commercially available injection pens. Because the housing components of an injection pen are generally tubular and elongate having a generally circular cross-section, the parting mechanism acts to separate the injection pen housing circumferentially relative to the axis AA shown in FIG. 1.

In some embodiments, the parting mechanism includes a cutter, such as a cutting wheel, cutting blade, or cutting wire, for example. The specific form and material composition of the cutter depends on the material of the injection pen housing. Currently available injection pen housings are typically formed from plastic. Consequently, the cutter form and composition is selected appropriately and will be discussed in further details herein below. Injection pens with housing materials other than plastic may be introduced in the future. Thus, a cutter suitable for use on materials such as aluminum or other metals, wood, bamboo, or bagasse, or composites is included, in some embodiments.

In some embodiments, the parting mechanism applies the cutting edge of the cutter to the injection pen cartridge holder orthogonal to axis AA under a controlled force. The parting mechanism limits the force applied by the cutter to the cartridge holder to avoid fracturing the cartridge holder or breaking or damaging the cartridge within. The fixture mechanism allows for a rotational movement of the injection pen about axis AA to facilitate a circumferential cut through the cartridge holder. As most injection pen cartridge holders are not uniformly round, during the rotation the parting mechanism accommodates a changing distance between the centerline of rotation and the cutter edge. To increase effectiveness of the parting mechanism an additional cutting means is provided, such as low or high (i.e., ultrasonic) vibration or heat, in some embodiments.

In some embodiments, the parting mechanism includes a cutting saw formed as a serrated disk or a serrated blade. In still another form, the parting mechanism includes an abrasive mechanism, such as an abrasive disk, abrasive blade, file, or abrasive wire. In still another form the parting mechanism may employ heat to part the cartridge holder, such as through a hot knife, hot wire, or ultrasonic knife. In still another form, the parting mechanism may include a roller mechanism which applies a force transversely to the injection pen housing sufficient to structurally weaken the housing in such a way that the regions on either side of weakened region can be pivoted or twisted with respect to each other sufficient for removal of the medicament cartridge. In another form, the parting mechanism may employ a roller mechanism which applies a force transversely to the injection pen housing at an overlapping area between the cartridge holder and pen body such that a mechanical coupling, weld, adhesive, or other bond between the cartridge holder and pen body is sufficiently disrupted for the cartridge holder and pen body to be separated for removal of the medicament cartridge.

The parting mechanism or fixing mechanism may include a means for countering the force applied by the parting mechanism to the injection pen housing such that the net bending stress on the injection pen is limited, thereby preventing breakage, or otherwise damaging the injection pen housing in an unprescribed manner, and/or breaking or otherwise damaging the medicament cartridge, in some embodiments.

The parting mechanism is accessed by the user for maintenance or replacement of components that are worn, broken, or have otherwise reached the end of useful life, in some embodiments. The parting mechanism may be of a modular design such as a cartridge, to facilitate ease of replacement. Likewise, other components of the recycler may be of a modular design to facilitate disassembly and recycling of the injection pen at end of life.

The injector disassembly tool housing contains the fixing mechanism and the parting mechanism. The housing may comprise several sections which can be reversibly assembled and disassembled by the user to access the fixing mechanism for ease of injection pen insertion and removal, and for engaging and disengaging the parting mechanism. In some embodiments, the housing provides for the operation of the disassembly device such that injection pen disassembly occurs in a controlled, well-defined, prescribed manner by maintaining a desired spatial relationship between fixing mechanism and parting mechanism. The housing may additionally provide a means for movement of the fixing mechanism and injection pen relative to the parting mechanism to facilitate the action of the parting mechanism.

Some embodiments of the disassembly device will be operated manually for users desiring a simpler, less costly handheld system. Some embodiments include an electro-mechanical system for use in settings wherein large numbers of injection pens are to be recycled.

Because the injection pen is not designed for cartridge replacement, disassembly of the injection pen with the injector disassembly tool renders the injection pen unusable.

After the at least partial disassembly of the injection pen and removal from the fixing mechanism, the medicament cartridge can be removed from the remainder of the injection pen. Thereafter, the medicament cartridge is recycled or disposed of separately from the remaining components of the injection pen. Optionally, the injector disassembly device additionally includes a parting mechanism for removing the metal crimp cap and the elastomeric septum and piston from the drug cartridge in some embodiments, thereby allowing any remaining medicament and potential biological waste to be removed making recycling of the drug cartridge barrel safer and more convenient.

Also disclosed are methods for disassembly of an injector pen in a controlled, well-defined, prescribed manner. In some embodiments, steps of such methods include inserting an injection pen into an injector disassembly tool; fixing the injection pen in the tool; engaging a parting mechanism to divide the housing into at least two pieces or partially divide or otherwise weaken the housing; releasing the injection pen from the tool; separating the divided housing pieces, or pivoting or twisting the regions of the housing on either side of the region weakened in the engaging step; removing the cartridge from the cartridge holder; and recycling the cartridge separately from the remainder of the injection pen. In this way even a disposable medicament injection pen intended to be discarded in landfills or incinerated after use can be made available to more environmentally sensitive disposal or recycling methods.

The foregoing and other features and advantages of the example embodiments will be apparent to those of ordinary skill in the art from the following more particular description of the invention and the accompanying drawings.

### BRIEF DESCRIPTION OF THE DRAWINGS

FIG. 1 is a perspective view of prefilled multidose disposable prefilled pen injector representative of the prior art;

FIG. 2 is a partially exploded view of a prefilled multidose disposable pen injector representative of the prior art;

FIG. 3 is a perspective view of a pen injector disassembly tool;

FIG. 4 is a cutaway view of a pen injector disassembly tool;

FIG. 5 is a cutaway view of pen injector disassembly tool containing a pen injector;

FIG. 6 is a partially exploded view of a pen injector disassembly tool;

FIG. 7 is an exploded view a pen injector disassembly tool;

FIG. 8 is a partially exploded view of a cutter wheel cartridge of a pen injector disassembly tool;

FIG. 9A is a front perspective view of a cutter cam of the cutter wheel cartridge shown in FIG. 8;

FIG. 9B is a rear perspective view of the cutter cam of FIG. 9A;

FIG. 10 is a partially exploded view of a pen injector disassembly tool containing a pen injector;

FIG. 11A is a front perspective view of an alternative embodiment of a pen injector disassembly tool coupled to a pen injector;

FIG. 11B is a rear perspective view of an alternative embodiment of a pen injector disassembly tool coupled to a pen injector;

FIG. 12A is a perspective view of a second alternative embodiment of a pen injector disassembly tool coupled to a pen injector;

FIG. 12B is a perspective view of a second alternative embodiment of a pen injector disassembly tool coupled to a pen injector;

FIG. 13A is an additional perspective view of the second alternative embodiment of a pen injector disassembly tool coupled to a pen injector;

FIG. 13B is a cutaway view of the additional perspective view of a second alternative embodiment of a pen injector disassembly tool coupled to a pen injector;

FIG. 14A is a perspective view of an additional alternative embodiment of a pen injector disassembly tool coupled to a pen injector;

FIG. 14B is a partial cutaway view of the additional alternative embodiment of the pen injector disassembly tool of FIG. 14A coupled to a pen injector;

FIG. 15A is a perspective view of yet another additional alternative embodiment of a pen injector disassembly tool, shown partially assembled and coupled to a pen injector;

FIG. 15B is a second perspective view of the yet another additional alternative embodiment of the pen injector disassembly tool shown in FIG. 15A, shown partially assembled and coupled to a pen injector;

FIG. 16A is a perspective view of a separate additional example embodiment of a pen injector disassembly tool coupled to a pen injector;

FIG. 16B is an exploded view of the separate additional example embodiment of a pen injector disassembly tool shown in FIG. 16A coupled to a pen injector;

FIG. 17A is a perspective view of a fixing mechanism of a pen injector disassembly tool; and

FIG. 17B is a partially exploded view of the fixing mechanism shown in FIG. 17A.

### DETAILED DESCRIPTION

Various example embodiments of a pen injector disassembly tool and methods of use are described in detail herein. Some definitions will be set forth, followed by a brief discussion of existing pen injectors presented to contribute to the detailed description of example embodiments of a pen injector disassembly tool which follow.

As used herein, “distal” refers to a position relative to an end of a pen injector at where an injection means, such as a needle, is disposed. As used herein, “proximal” refers to a position relative to an end of a pen injector opposite a distal end. The distal portion of a pen injector is nearest the skin injection site. Two segments of a pen injector may be positionally compared to one another by stating that one segment or component is either proximal or distal to the other segment or component.

As used herein, “front” and “rear” refer to a position relative to an end of a pen injector disassembly tool, wherein “front” refers to the tool end nearest the injection needle location of a pen injector coupled to the tool and “rear” refers to the tool end opposite the injection needle location of a pen injector coupled to the tool. Where “front” and “rear” are used with respect to a pen injector disassembly tool or any component part of such a tool, these terms related to the position of the tool or component part with respect to a pen injector inserted in the tool, regardless of whether the tool being described contains the pen injector.

Further, any directional references as used herein, such as right, left, up, down, top, bottom, and the like are intended for convenience of description and do not limit the disclosed structures to any particular positional or spatial orientation.

As used herein, “axial” or “axially” refers to a direction parallel to a central longitudinal axis of a structure, such as a pen injector of a pen injector disassembly tool.

As used herein, “radial” or “transverse” refers to a direction orthogonal to a central longitudinal axis of a structure.

As used herein, “circumferential” or “circumferentially” refers to a curved path around the body of a structure or sub-structure in a plane orthogonal to a central longitudinal axis.

As used herein, “additional embodiment,” “another additional embodiment,” “yet another additional embodiment,” “separate additional embodiment,” and similar terms refer to different examples of embodiments of pen injector disassembly tools within the scope of the disclosures and teachings found herein, and the components thereof.

Attention will now be directed to providing detailed descriptions of example embodiments of an injector disassembly tool and methods of with reference to the several drawing figures.

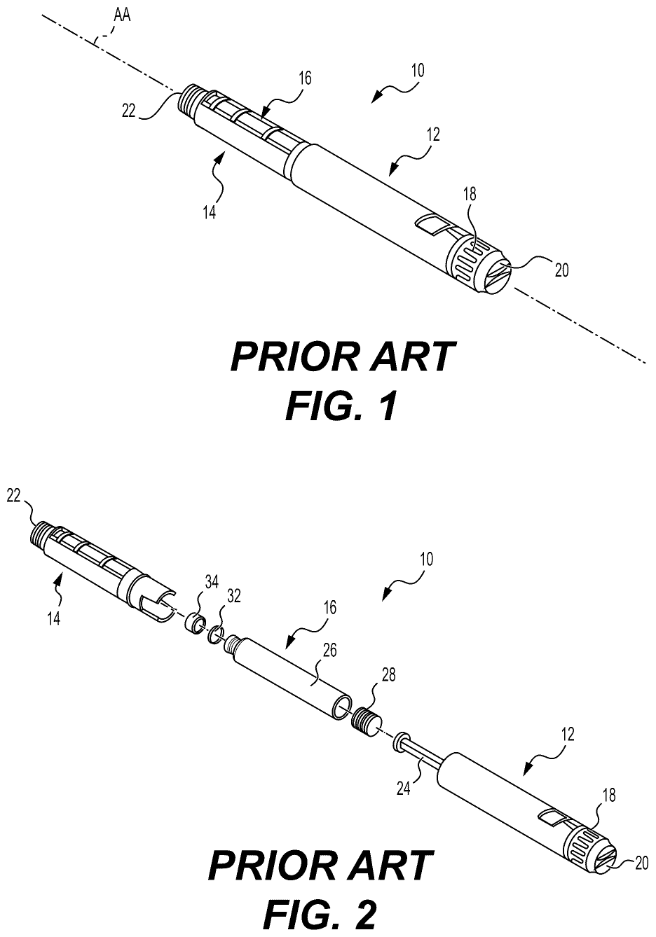

FIG. 1-2 are representative of the prior art. FIG. 1 is a perspective view of a prior art prefilled multidose disposable pen injector and FIG. 2 is a partially exploded view of a prior art prefilled multidose disposable pen injector.

FIG. 1 shows a pen injector 10 typical of a disposable, multidose pen injector found in the prior art. Pen injector 10 is representative of a multidose pen injector typically used by a patient to self-administer a medication, such as insulin for a patient with diabetes. Pen injector 10 generally presents an elongated somewhat cylindrical formed disposed about a central longitudinal axis AA.

Pen injector 10 includes a pen body 12 disposed proximal to a cartridge holder 14. Pen body 12 and the cartridge holder 14 are either formed or inseparably coupled into a unitary body, such as by injection molding or coupling with a fixed mechanical fastener, welding, adhesive, or the like, for example. Contained within cartridge holder 14 is a cartridge 16 pre-filled with a liquid medicament. Cartridge 16 is visible through an opening in cartridge holder 14, depicted in FIG. 1 as a slot with supporting ribs. Some manufacturers of prior art pen injectors construct cartridge holders from transparent plastic, obviating the need for an opening.

FIG. 2 shows an exploded view of pen injector 10. Cartridge 16 is typically in the form of an elongated glass barrel 26 having an elastomeric septum 32 at its distal end. Septum 32 is held in place by a metal crimp cap 34 in many prior art embodiments. An elastomeric piston 28 is disposed within a proximal end of barrel 26 and forms a fluid seal with barrel 26 but is free to move along long axis AA. Pen body 12 houses a dosing mechanism and a driver mechanism for expelling the medicament. The user can rotate a dosing knob 18 to set a desired volume of medicament to be injected. The user then initiates the injection by depressing an injection button 20, which is most commonly located at the proximal end of the pen injector. Injection button 20 actuates the driver mechanism, such as by distally advancing a jack screw 24. The advancing jack screw 24 causes the displacement of piston 28 which, in turn, displaces the medicament, expressing the medicament out of cartridge 16 through a pen needle (not shown) at the distal end of injector 10. Distal threads 22 for securing the pen needle (not shown) are typically disposed at the distal end of cartridge 16. Following operation, the pen needle is removed.

Some manufacturers have developed twist-lock style connections between cartridge holder 14 and a compatible pen needle. When secured, the proximal end of the pen needle pierces septum 32 thereby establishing a fluid communication between the pen needle and the medicament within cartridge 16. The distal portion of the pen needle is configured to pierce the skin, allowing the medicament to be expressed into the skin, subcutaneous tissue, or muscle of the patient. The pen needle is designed to be used only once and is to be replaced by the user after each injection.

Pen injector 10 is representative of multiple dose disposable medicament pen injectors, such as those commonly used for the injection of insulin. The specific designs of each manufacturer's insulin pen injectors generally include cartridge holders, bodies, dose knobs, and injection buttons of different lengths and cross-sectional profiles. Nearly all available insulin pens share an International Standards Organization (ISO) standard thread at the distal end compatible with available pen needles.

Pen body 12, internal components, dose knob 18, injection button 20, jack screw 24, and cartridge holder 14 are most commonly formed from plastic whereas cartridge 16 comprises glass, piston 28 comprises a first synthetic elastomeric material, septum 32 comprises a second elastomeric synthetic material, and crimp cap 34 comprises a metal. Any potential biological waste in the form of body fluid or tissue which enters pen injector 10 through the pen needle is contained within cartridge 16. If pen injector 10 could be easily, rapidly, and safely disassembled in such a way as to separate cartridge 16 without breaking the containment of the drug or biological material by breaking or otherwise damaging glass barrel 26, the remaining plastic portions of pen injector 10 could be recycled. In addition, if cartridge 16 itself could be further disassembled by separating crimp cap 34, septum 32, piston 28, the glass could also be recycled separately.

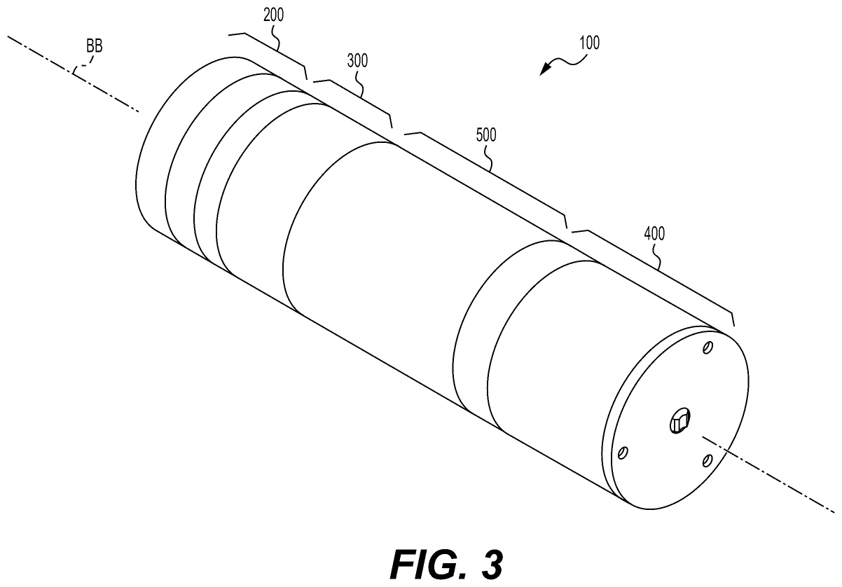

FIG. 3 is a perspective view of a pen injector disassembly tool. FIG. 3 shows an injector disassembly tool 100 comprising a penholder section 200, a cutter section 300, a drive section 400, and a coupler section 500. Tool 100 presents an elongated, generally cylindrical shape having a central longitudinal axis BB. Disassembly tool 100, as shown in FIG. 3, is an example embodiment of a disposable injector disassembly tool configured for the disassembly of a prefilled, multidose disposal injection pen. Sections 200, 300, 400, and 500 are coupled together sequentially in the coaxial axial relationship shown in FIG. 3, in some embodiments.

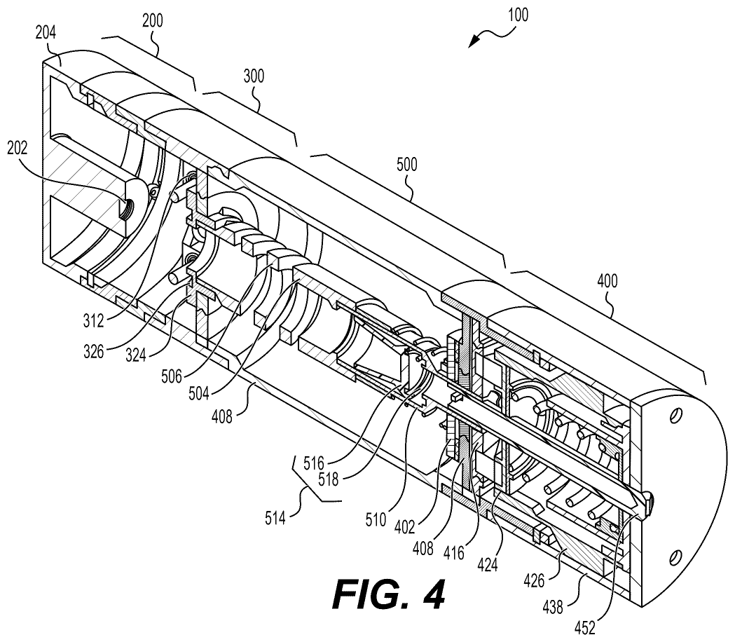

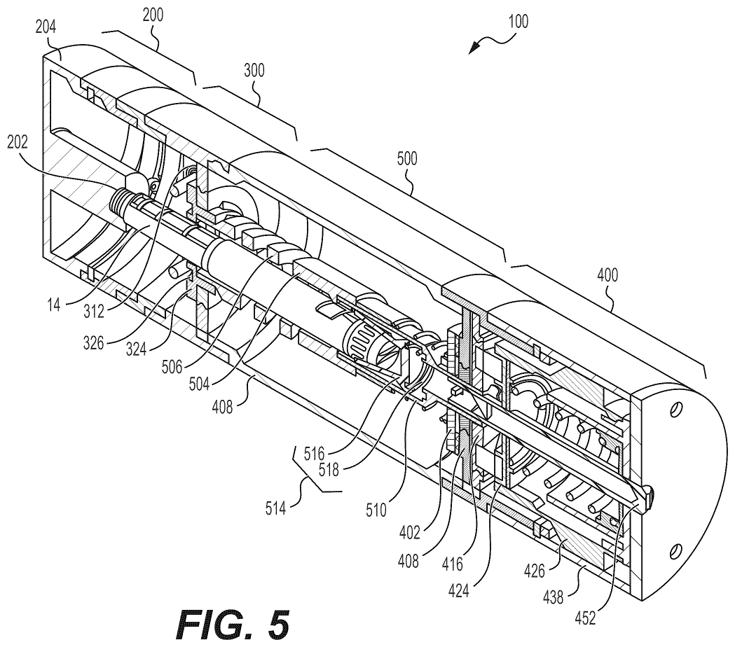

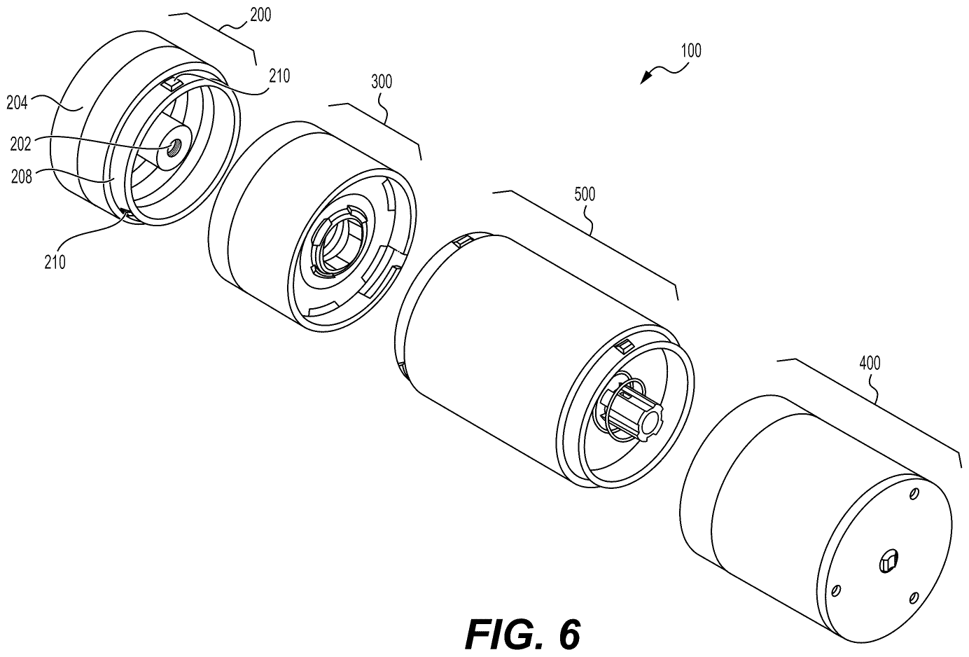

FIG. 4-5 are cutaway views of a pen injector disassembly tool. FIG. 4 shows a sectional view taken along axis BB of the pen recycler 100 example embodiment shown in FIG. 3. FIG. 5 show the same longitudinal section of tool 100 with pen injector 10 fixed into position within tool 100. In this fixed position, pen injector 10 is seen to be coaxial with tool 100 with respect to BB, as shown in FIG. 5.

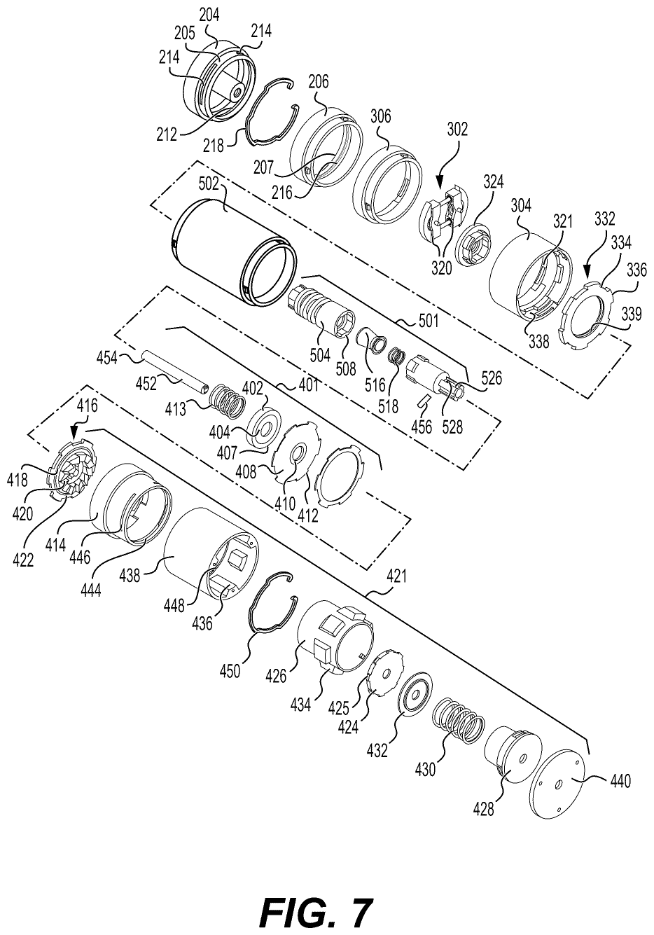

Stepped changes in the wall thickness of the various housing sections, in some embodiments, allow the sections to overlap slightly when fit together, maintaining a generally uniform cylindrical shape of injector disassembly tool 100 when section 200, section 300, section 400, and section 500 are fully assembled. Stepped diameters in housing sections are overlapped in a “male-inside-female” arrangement with interaction of complementary surface features disposed on each step to reversibly join together the several sections of injector disassembly tool 100. These overlapping stepped changes in housing sections is shown in FIG. 4-5. Several configurations of interlocking surface features known in the art may be employed for joining the housings of each section. For example, a twist-lock mechanism wherein a ramped protrusion is passed through a corresponding opening by twisting two sections about axis BB in relation to one another to lock the sections together after mating the inner step (male) of one section with the outer step (female) of the adjoining section. A male end of the housing includes an inner step 208 presenting a plurality of surface features 210 disposed circumferentially around inner step 208. Inner s8ep 207 is configured to insert into an outer step of an adjacent section, such as outer step 306 of cutter section 300 shown in FIG. 7, wherein a plurality of protrusions 210 interact with a corresponding number of L-shaped slots circumferentially disposed in outer step 306. When mated together, twisting either of section 200 or section 300 with respect to one another about axis BB locks together pen holder section 200 and cutter section 300. In some other embodiments, injector disassembly tool 100 comprises a continuous cylindrical housing formed from some or all of combined section 200, section 300, section 400, and section 500, in any combination. In some embodiments, section 200 and section 300 are joined permanently using mechanisms such as mechanical fastening, plastic welding techniques, insert molding, in mold assembly, snap fits, adhesives, or any other joining method common in the art.

FIG. 6 is a partial exploded view of a pen injector disassembly tool. FIG. 6 shows injector disassembly tool 100 comprising the generally cylindrical sections of an injector disassembly tool 100 coupled together end-to-end.

In some embodiments, pen holder section 200 includes a base 204 with a female threaded fixture 202. Threaded fixture 202 is configured to receive male distal threads 22 of pen injector 10.

Cutter section 300 comprises a mechanism for separating a medicament cartridge from injection pen 10 and drive section 400 comprises a torque limiting driver for actuating parting mechanism of cutter section 300, in some embodiments. Coupler section 500 comprises a shaft configured to directly transfer torque from drive section 400 to the parting mechanism of cutter section 300. A shaft of coupler section 500 comprises a self-centering live center fixture that removably couples with dosing knob 18 and injection button 20 of pen injector 10. These components of holder section 200, cutter section 300, drive section 400, and coupler section 500 are discussed in further detail herein below.

FIG. 7 is an exploded view of a pen injector disassembly tool. FIG. 7 shows an exploded view of injector disassembly tool 100. In some embodiments, pen holder base 204 of section 200 forms outer step 205 at the proximal end of base 204. Outer step 205 couples with corresponding inner step 207 of a rotary coupler 206, allowing the base 204 and coupler 206 to overlap slightly while maintaining the generally uniform cylindrical shape of injector disassembly tool 100. An internal circumferential first groove 212 having a plurality of through slots 214 is disposed on outer step 206, in some embodiments. A corresponding second groove 216 is disposed on rotary coupler 206 in radial alignment with first groove 212 in a configuration whereunder base 204 and rotary coupler 206 are joined. A first retaining ring 218 seats in first groove 212, protrudes slightly through slots 214, and interacts with second groove 216. In this manner, base 204 and rotary coupler 206 lock together axially, allowing for relative rotation of base 204 with respect to rotary coupler 206 around axis BB. Wherein injector pen 10 is fixtured in injector disassembly tool 100 as shown in FIG. 5, pen injector 10 can be rotated about axis BB by rotating base 204. Rotary coupler 206 is coupled to a cutter retainer 306 using the previously described coupling mechanism to join pen holder section 200 with cutter section 300, in some embodiments.

Cutter section 300 comprises components configured to cut a housing of pen injector 10 to facilitate separation of pen injector 10 components and removal of its medicament glass cartridge. Cutter section 300 comprises a cutter wheel cartridge 302 configured to insert into a cutter housing 304. Frame 320 displays tabs that fit within corresponding slots 321 on housing 304, in some embodiments. A cutter retainer 306 couples to cutter housing 304 to fix cutter cartridge 302 within cutter housing 304, in some embodiments.

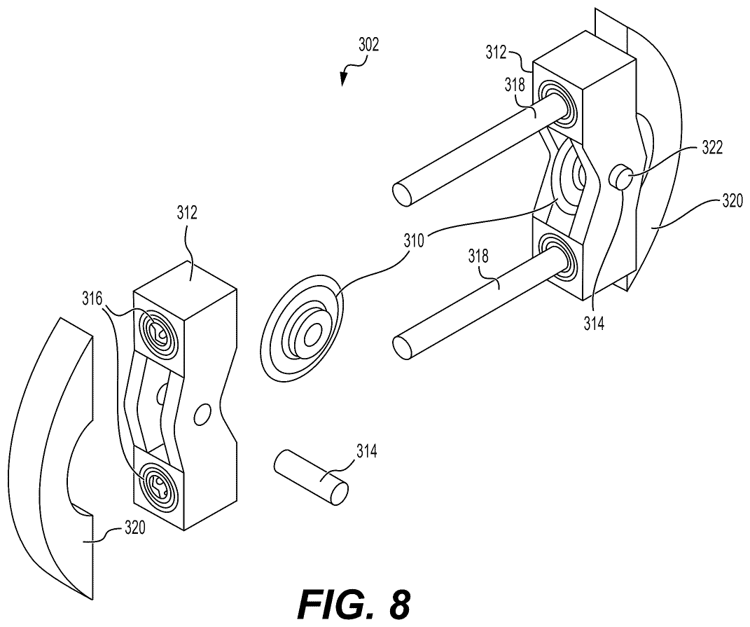

FIG. 8 is a partial exploded view of a cutter wheel cartridge of an injector disassembly tool. FIG. 8 shows cutter wheel cartridge 302 with two, diametrically opposed cutter wheels 310, each held within a carrier 312 by a shaft 314. Carrier 312 is fitted with a linear bearing 316 that rides on a pair of parallel guide rails 318 anchored in the frames 320, as shown, in some embodiments. Each carrier 312 is free to move independently along guide rails 318 while keeping cutting wheels 310 in a plane perpendicular to axis BB. In some embodiments, each axle shaft 314 includes a shaft protrusion 322 extending slightly beyond the surface of carrier 312, as shown by FIG. 8 which functions to mount axle shaft 314 within a cutter cam 324.

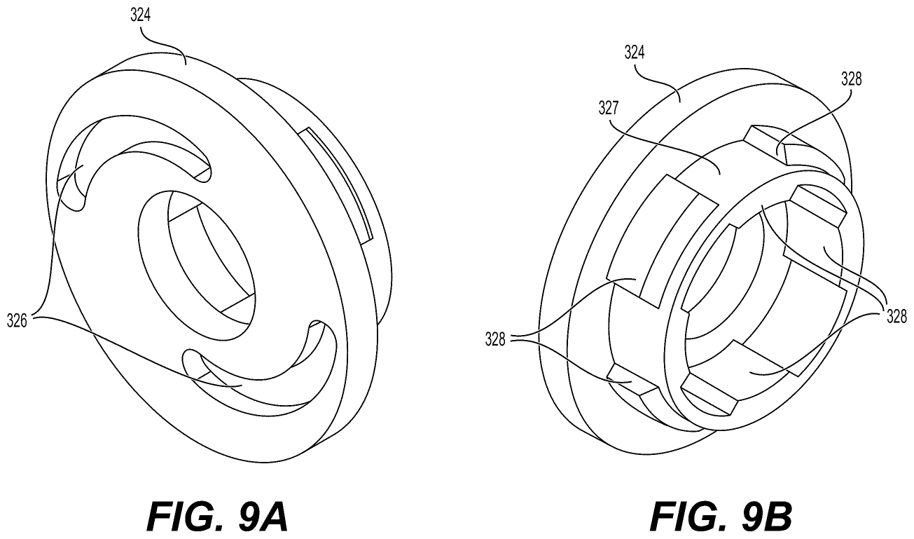

FIG. 9A is a front perspective view of a cutter cam of the cutter wheel cartridge shown in FIG. 8. FIG. 9B is a rear perspective view of the cutter cam shown in FIG. 9A. FIG. 9A-B show cutter cam 324. Cutter cam 324 comprises a formed, multi-tiered discoid body having two through slots 326, in some embodiments. Each slot 326 receives an axle shaft protrusion 322. A curvature of slots 326 is configured wherein by rotating cutter cam 324 about axis BB, the distance between cutter wheel carriers 312 and hence cutter wheels 310 varies. When pen injector 10 is positioned within injector disassembly tool 100 as shown in FIG. 5, rotation of cutter cam 324 urges cutter wheels 310 into engagement with an outer surface of pen injector 10 fixed within injector disassembly tool 100.

FIG. 9B shows cutter cam 324 forming a unitary body with a hollow hub 327. Hub 327 is configured with a plurality of inner splines 328 and a plurality of outer splines 330. Referring back to FIG. 7, cutter cam 324 interacts with a cutter cam bearing 332 fit with an outer race 334 and having a plurality of tabs 336 positioned around an outer circumference of bearing 332. Tabs 336 engage with corresponding slots 338 in cutter housing 304, wherein outer race 334 is fixed in position relative to cutter housing 304. An inner race 339 of cutter cam bearing 332 is keyed to fit the outer splines 330 of cutter cam 324. Cutter cam bearing 332 retains cutter cam 324 in axial alignment while allowing cutter cam 324 to rotate about axis BB.

As shown in FIG. 7, coupler section 500 comprises a coupler housing 502 enclosing a shaft section assembly 501, in some embodiments. Coupler housing 502 couples with cutter housing 304 through a reversible interaction, in some embodiments, using the example “twist lock” mechanism described herein above. Whereunder section 300 and section 500 are coupled, coupler housing 502 retains cutter cam bearing 332 within cutter housing 304. A plurality of inner splines 328 of cutter cam 324 are keyed to fit the splines on the distal end of a hollow flex shaft 504 contained within coupler housing 502 as shown in FIG. 7.

Shaft section assembly comprises a hollow flex shaft 504, inner splines 508, a drive shaft 510, outer splines 512, cone 516, and compression spring 518, in some embodiments. Flex shaft 504 is fitted with torsion spring 506 which provides potential energy in the form of stored torque for transfer to flex shaft 504, wherein relative rotation about axis BB of cutter section 300 with respect to coupler section 500 generates tension in spring 506 (shown in FIG. 4-5). Upon release of the stored spring tension, flex shaft 504 has a resiliency to return to its pre-tensioned axial position. Flex shaft 504 couples to a hollow drive shaft 510. A proximal end of the flex shaft 504 comprises a plurality of inner splines 508. Inner splines 508 are keyed to engage with a plurality of outer splines 512 disposed on an outer surface of drive shaft 510. A self-centering live center 514 mounts between flex shaft 504 and drive shaft 510 in a configuration whereunder flex shaft 504 and drive shaft 510 are coupled. Live center 514 couples to the proximal end of pen injector 10. Live center 514 comprises a hollow cone 516 and a compression spring 518, as shown in FIG. 7 and FIG. 5, for example. As seen in FIG. 5, cone 516 receives dosing knob 18 of pen injector 10 maintaining pen injector 10 long axis AA coaxial with axis BB of injector disassembly device 100. Cone 516 is free to both translate along axis BB and also rotate within coupler housing 502 and cutter housing 304 wherein cutter section 300 and coupling section 500 are coupled together. Compression spring 518 allows injector disassembly tool 100 to accommodate a range of pen injector 10 lengths and dosing knob 18 diameters.

Drive section 400 comprises a torque-limiting driver assembly 421 and an anti-reverse ratchet 401, as further shown in FIG. 7. A pressure plate 416 of the torque limiting driver assembly 421 comprises an outer race 418 and an inner race 420. A plurality of tabs 403 circumferentially disposed around an outer circumference of pressure plate 410 engage with corresponding slots 405 disposed circumferentially around an inner surface of a drive coupler 414. Tabs 403 and slots 405 axially and rotationally fix outer race 418 with respect to drive coupler 414. Inner race 420 comprises a central splined keyway configured to receive outer splines 526 of drive shaft 510. Rotation of shaft section 501 and cutter cam 308 is coupled to the rotation of inner race 420 wherein the effective cutting diameter of cutter cartridge 302 is varied, the cutting force that is applied to the injection pen cartridge is varied, or both the effective cutting diameter and the applied cutting force is varied.

Torque limiting driver assembly 421 limits the amount of torque which can be applied to shaft section 501 such that a torque force applied by cutter wheels 310 to an outer surface of a cartridge holder of pen injector 10 is limited to a magnitude sufficient to divide the cartridge holder at least partially without risking breakage of the glass medicament cartridge contained therein. The design and operation of torque limiting driver assembly 421 is known in the art. Pressure plate 416 also comprises a one-way ramping gear 422 having teeth configured to mesh with corresponding clutch plate gear teeth 425 of a clutch plate 424. Clutch plate 424 comprises tabs disposed circumferentially which fit into a corresponding plurality of slots in a clutch drum 426 having clutch drum slots. The clutch drum slots are axially elongated allowing axial translation of clutch plate 424 during rotation of coupled clutch plate 424 and clutch drum 426. A drum cap 428 holds a clutch spring 430 in compression against a spring retainer 432 in contact with a proximal surface of clutch plate 424. Clutch drum 426 comprises splines 434 that engage with corresponding handle splines 436 within a handle 438. Clutch drum 426 and drum cap 428 are retained within handle 438 by a handle cap 440. Consequently, operation of torque limiting driver assembly 421 by rotating handle 438 causes rotation of clutch plate 424 and engaged pressure plate 416.

The torque limiting characteristic of torque limiting driver assembly 421 derive from a controlled slippage of the one-way ramped surface of gear teeth 425 of clutch plate 424 on the one-way ramped surface of gear teeth 422 of pressure plate 416. As the torque transmitted to shaft section 501 increases beyond a controlled limit, the slippage overcomes the compressive force of the clutch spring 430 causing the clutch plate to rotate one gear position relative to the pressure plate. The clutch spring 430 then causes clutch plate 424 to move axially in a distal direction, snapping back to full engagement with the pressure plate 416 with an audible click, in some embodiments. Further rotation of the handle 438 results in additional slippage of clutch plate 424 accompanied by more clicks. As such, the applied torque to the shaft section 501 reaches a predefined limit.

Drive coupler 414 couples to coupler housing 502 using a coupling means, such as the twist lock mechanism described herein above. The proximal end of the drive coupler 414 overlaps with the handle 438. Drive coupler 414 comprises an internal circumferential first groove 444 disposed along an inner surface of drive coupler 414 and a plurality of through slots 446. Handle 438 has a second groove 448 corresponding and in radial alignment with first groove 444 when driver coupler 414 and handle 438 are coupled. A second retaining ring 453 seats in first groove 444 and protrudes slightly through slots 446 and to also seat in second groove 448. This interaction of second retaining ring 453 axially locks drive coupler 414 and handle 438 together but allows for rotation of handle 438 relative to drive coupler 414 around axis BB.

Torque applied to shaft section 501 is maintained during slippage of torque limiting driver assembly 421 by anti-reverse ratchet assembly 401. The design and operation of an anti-reverse ratchet is known in the art and the description herein is by example only. A star ratchet gear 402 with internal splines 404 is keyed to fit over external splines 526 of drive shaft 510. The internal splines 404 and external splines 526 interact such that, when assembled, drive shaft 510 and star ratchet gear 402 rotate together but the start ratchet gear 402 may freely translate axially. Star ratchet gear 402 includes one-way ramped teeth 407 disposed circumferentially around a proximal-facing surface. Teeth 407 mesh with corresponding one-way ramped teeth on star ratchet hub 408. Star ratchet hub 408 includes tabs 412 circumferentially disposed which interact with corresponding through-slots on drive coupler 414, wherein star ratchet hub 408 is rotationally fixed relative to drive coupler 414. Star ratchet hub 408 also includes a bearing 410 having internal splines keyed to accommodate external splines 526 of drive shaft 510. Bearing 410 allows rotation of drive shaft 510 relative to star ratchet hub 408.

A compression spring 413 interposed between drive shaft 510 and star ratchet gear 402 maintains engagement of the teeth of star ratchet gear 402 and star ratchet hub 408. The ramp on the ratchet gear teeth 407 and the compressive force of the spring 413 are configured such that slippage between star ratchet gear 402 and star ratchet hub occurs at lower torque magnitude on drive shaft 510 than the torque magnitude required to cause slippage of torque limiting driver assembly 421. Because star ratchet hub 408 is rotationally fixed, relative rotation of star ratchet gear 402 requires slippage on star ratchet hub 408. Slippage of star ratchet gear 402 is accompanied by audible clicks as spring 404 snaps star ratchet gear 402 into full engagement with star ratchet hub 408. During full engagement of star ratchet gear 402 and star ratchet hub 408, relative rotation between these structures is restricted to the direction of slippage because star ratchet hub 408 is fixed to drive coupler 414 and the corresponding teeth are in abutment.

As the ramp of ratchet gear teeth 407 on star ratchet gear 402 is opposite that of the corresponding teeth on clutch plate 424, slippage of star ratchet gear 402 occurs in the direction of rotation of handle 438. Further, handle 438 is limited to rotation in only one direction when star ratchet gear 402 and star ratchet hub 408 are fully engaged. Therefore, as handle 438 is rotated causing torque on shaft section 501 to increase, handle 438 can be released, and anti-reverse ratchet assembly 401 will maintain torque on shaft section assembly 501.

Anti-reverse ratchet 401 assembly also comprises a star ratchet gear 402 release mechanism to disengage star ratchet gear 402 from star ratchet hub 408 wherein any stored torque is released. The resiliency of flex shaft 504 allows shaft 504 to rotate counter to the slippage direction as it returns to its resting state. This has the effect of releasing force application of cutter wheels 310 on pen injector 10. In some embodiments, the release mechanism employs a button 452 which fits in the hollow distal end of drive shaft 510. Button 452 includes a keyway 454 configured to accommodate a key 456. Drive shaft 510 includes an axially elongated key slot 528 disposed transversely. Key 456 has a length sufficient to extend radially beyond drive shaft 510 when inserted into the key slot 528. Star ratchet gear 402 is disposed on drive shaft 510 distal to the key. The shape and position of slot 528 allows star ratchet gear 402 to remain engaged with star ratchet hub 408 and key 456 to retain button 452. Depressing button 452 in the distal direction disengages star ratchet gear 402 from star ratchet hub 428. Upon release of button 452, star ratchet gear 402 returns to full engagement with star ratchet hub 408.

The direction of slippage as depicted in this example embodiment is such that handle 438 can only be rotated in a clockwise direction when injector disassembly tool 100 is viewed from the proximal end. It should be noted that the choice of direction of slippage is arbitrary. Other embodiments employing anticlockwise rotation of the handle 438 are envisioned.

### Methods of Use

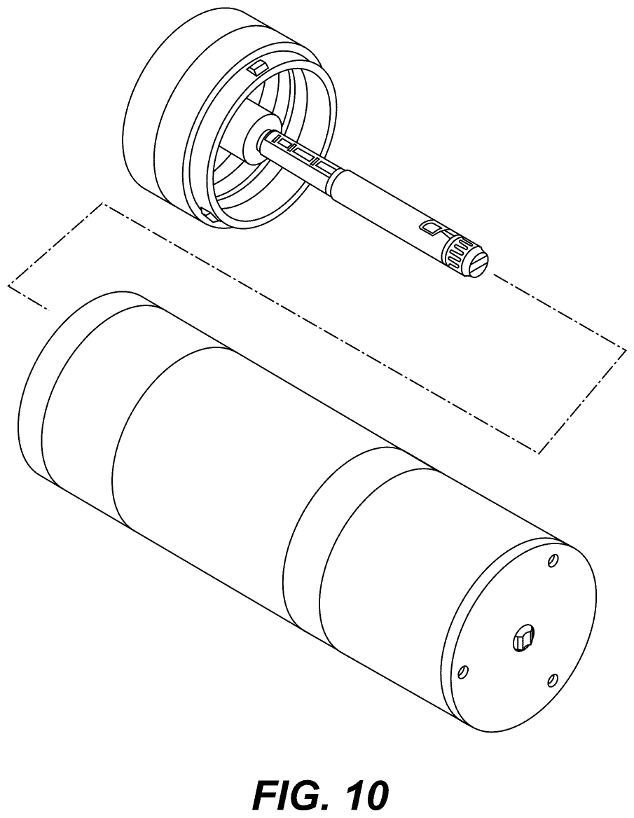

Methods of use of an injector disassembly tool offered in the example embodiments described herein above comprises the following steps:
Inserting—Pen injector 10 is inserted in pen holder section 200. Pen holder section 200 is decoupled from the remainder of disassembly tool 100 and distal threads 22 of pen injector 10 are threaded into threaded fixture of base 204. FIG. 10 is a partial exploded view of an injector disassembly tool. FIG. 10 shows pen injector 10 coupled to base 204 of pen holder section 200 separated from the assembled cutter section 300, coupler section 500, and drive section 400. FIG. shows disassembly tool following insertion of pen injector 10 during the inserting step.
Coupling—Base 204 bearing pen injector 10 is coupled to cutter section 300 by axially aligning base 204 and cutter section 300, approximating outer step 306 with inner step 208. FIG. 5 is a cutaway view of injector disassembly tool 100 containing pen injector 10 following the coupling step. As shown by FIG. 5, a proximal end of pen injector 10 is seated in cone 516 of live center 514. Spring 518 is under compression, helping to stabilize pen injector 10 and maintaining the proximal end from moving off axis BB. Pen injector 10 can be rotated within tool 100 in response to rotation of base 204.
Rotating—The user grasps and rotates handle 438 in a clockwise direction. Rotation is transferred to clutch drum 426 and clutch plate 424. Engagement of clutch plate 424 and pressure plate 416 transfers rotation of handle 438 to pressure plate 416. Rotation of pressure plate 416 causes rotation of drive shaft 510. Drive shaft 510 rotation initiates a ratcheting action between star ratchet gear 402 and ratchet hub 408. The ratcheting mechanism restricts rotation of drive shaft 510 to one direction, such as a clockwise direction in the example embodiments herein and prevents rotation in a counterclockwise direction. Drive shaft 510 transfers rotation to flex shaft 504, which, in turn causes rotation of cutter cam 324.

Shaft protrusions 322 on axles 314 of cutter wheel carriers 312 engage with slots 326 of cutter cam 324. As cutter cam 324 rotates, the rotation of slots 326 interact with shaft protrusions 326 to draw carriers 312 radially inward until cutter wheels 310 contact the external surface of cartridge holder 14 of pen injector 10. Continued rotation of handle 438 increases the inward radial force exerted on cartridge holder 14 by cutter wheels 31. Continued rotation of handle 438 also causes an increase of torque born by flex shaft 504. Torsion spring 506 of flex shaft 504 stores this torsional energy and allows some additional rotation of handle 438. Anti-reverse ratchet assembly 401 acting through star ratchet gear 402 and star ratchet hub 408 retains torsional energy of flex shaft 504 stored after handle 438 is released by the user.

Further continued rotation of handle 438 increases axial force on clutch plate 424 which eventually overcomes the compressive force of clutch spring 430 and clutch plate 424 slips on pressure plate 416. When this rotational slippage exceeds the width of a single ratchet gear tooth 407, clutch spring 404 snaps clutch plate 424 back into full engagement with pressure plate 416, causing an audible “click.” The click indicates to the user that further rotation of the handle will only result in additional slippage and the maximal stored torque in flex shaft 504 is reached as is the maximum inward radial force of cutter wheels 310 applied to cartridge holder 14.
Cutting—The user then grasps and rotates base 204 of pen holder section 200 causing rotation of pen injector 10 and inducing a parting action of cutter wheels 310 upon cartridge holder 14. As cutter wheels 310 part cartridge holder 14, the stored torque energy within flex shaft 504 allows rotation of cutter cam 324, keeping cutter wheels 310 in contact with sufficient inward radial force on cartridge holder 14 as cutter wheels 310 move deeper into the material of cartridge holder 14. Continued rotation of base 204 results in fully parting cartridge holder 14. A tactile change in resistance to rotation of base 204 indicates to the user that cutter wheels 310 are rotating freely on the glass surface of cartridge 16 within pen injector 10 and the parting of cartridge holder 14 is completed.
Releasing—Once the user determines that cartridge holder 14 has completely parted, the user depresses button 452 disposed on the end of the drive shaft 510, causing star ratchet gear 402 to disengage from ratchet hub 408, releasing the remaining torque on flex shaft 504 and removing the radial force of cutting wheels 310 from cartridge 16.
Decoupling—The user then twists holder section 200 with respect to section 300, decoupling holder section 200 from cutter section 300. The user then removes parted pen injector 10 from tool 100. The proximal parted end of pen injector 10 is separated from the distal parted end, allowing removal of glass cartridge 16 from the parted pen injector cartridge holder 14. The user then unscrews the distal portion of parted pen injector 10 from base 204. The plastic components of pen injector 10 can then be recycled, and the glass cartridge can be disposed of separately.

### Additional Embodiments

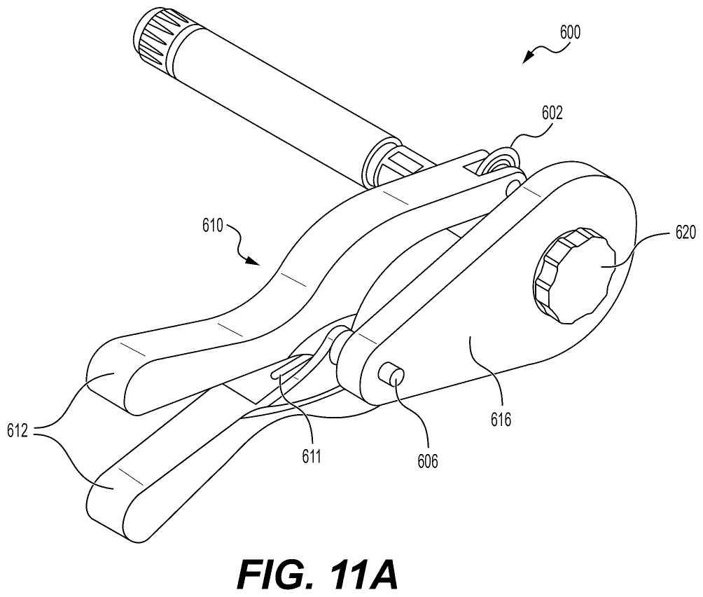

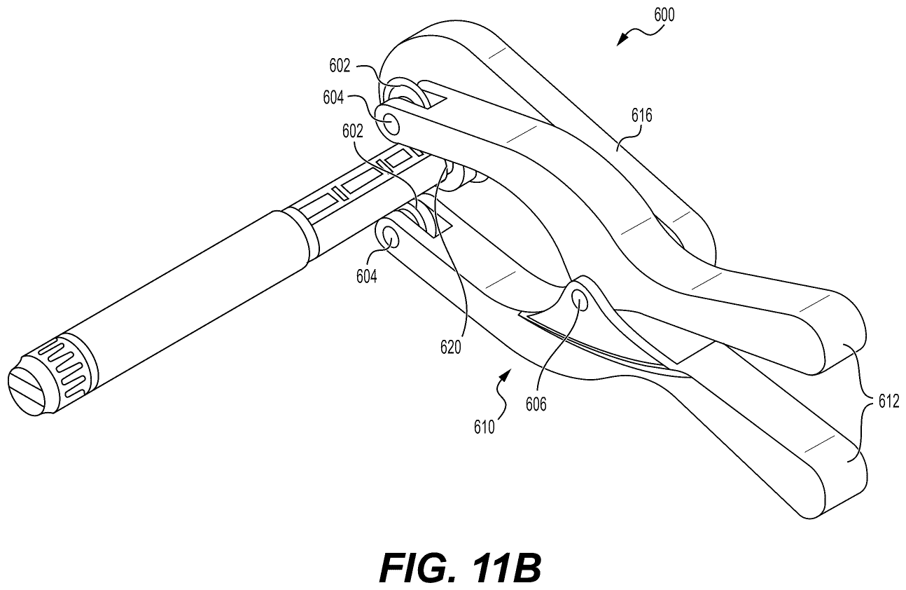

Additional embodiments of the injection pen recycler are shown in FIG. 11 through FIG. 17.

FIG. 11A is a front perspective view of an alternative embodiment of a pen injector disassembly tool coupled to a pen injector. FIG. 11B is a rear perspective view of an alternative embodiment of an injector disassembly tool coupled to a pen injector. FIGS. 11A-B show an injector disassembly tool 600. Injector disassembly tool 600 is shown loaded with a pen injector. Tool 600 comprises a spring clamp 610 having a torsion spring 611 and a pair of arms 612. The principle of design and operation of spring clamps is known in the art. Each of two arms 612 is fitted with a cutter wheel 602 rotatably mounted on an axle 604. A fixture plate 616 is joined to spring clamp 610 about a common pivot 606, as shown in FIGS. 11A-B. Pen injector 10 is coupled at male distal threads 22 to a knob 620 bearing a corresponding female threaded feature (not shown). The female threaded fixture of 620 is fixed to a shaft which runs through a bearing in the fixture plate and connects at its other end to knob 620. The parting action on pen injector 10 is accomplished by squeezing arms 612 together to transmit an inward radial force upon cutter wheels 602 contacting pen cartridge 10 and turning knob 620 to rotate the injection pen between the cutter wheels. The clamping force of the cutter wheels 602 on the cartridge holder induced by the torsion spring 611 allows the cutting wheels 602 to part the cartridge holder in a circumferential fashion as the knob is turned and the injection pen is rotated. The torsion spring 611 allows continuous force to be applied by the cutting wheels 602 as the effective cutting diameter changes during rotation of the injection pen.

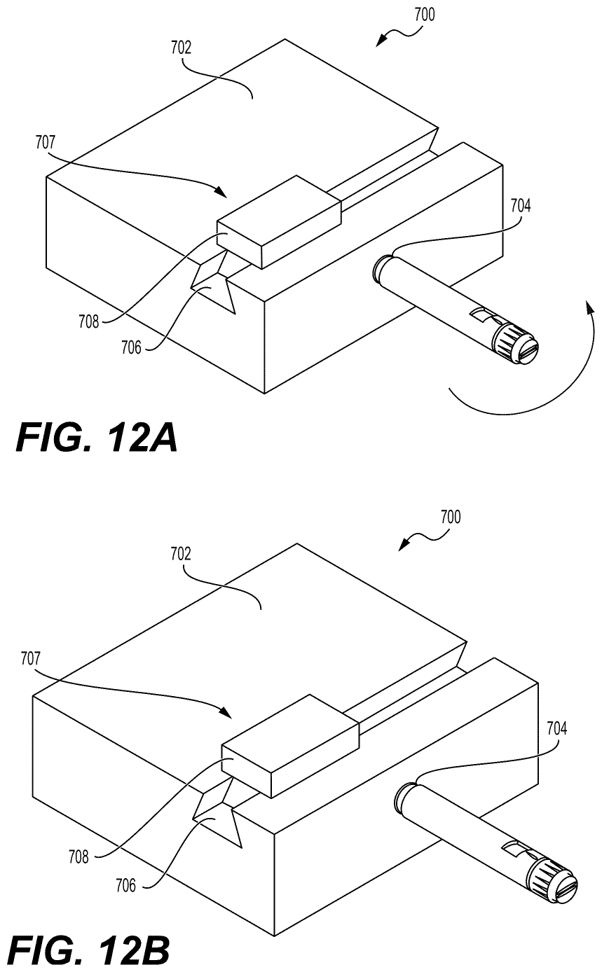

FIG. 12A and FIG. 12B show an additional example embodiment. An injector disassembly tool 700 comprises a base 702 fit with a generally cylindrical injection pen holder 704 for coupling to and fixing the injector in place. Base 702 is fitted with a guide slot 706, as shown in FIGS. 12A-B. Tool 700 additionally includes a blade guide 707 comprising a guide block 708 and a blade (not shown). Guide block 708 comprises a parting mechanism. Guide block 708 is keyed or otherwise configured to fit guide slot 706. Guide block 708 is itself fit with a slot to accommodate a cutting blade 710 which is held in place with a retaining pin 712.

Injection pen 10 is fixtured by inserting it into pen holder 704. Blade guide 707 is then inserted into guide slot 706 and slid to contact pen 10. Pen holder 704, guide slot 706 and guide block 708 have complementary shapes, as shown in FIGS. 12A-B, for example, configured such that the blade 710 contacts cartridge holder 14 of injection pen 10. The parting action of blade 710 is actuated by manually rotating the injection pen in the direction indicated by the circular arrow in FIG. 12A while applying moderate force on blade guide 708 in the direction indicated. Pen 10 is rotated until cartridge holder 14 is parted. Pen 10 is then removed from pen holder 704 and the glass cartridge separated from the remaining plastic for separate recycling and disposal.

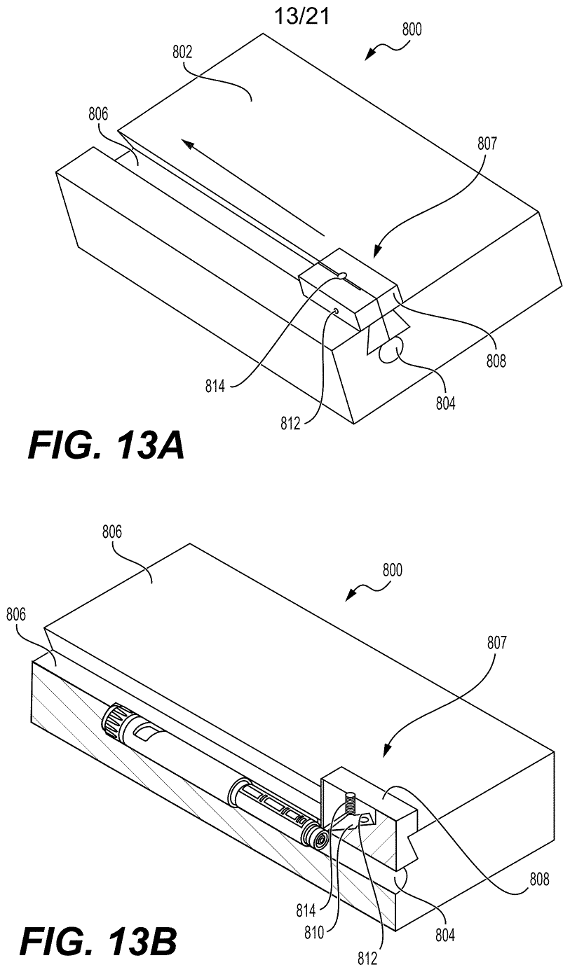

FIGS. 13A-B show an additional example embodiment of an injector disassembly tool 800. FIG. 13A is a top perspective view and FIG. 13B is a cutaway view. An injector disassembly tool 800 employs an axial cutting mechanism as opposed to the circumferential cutting mechanism for parting the injection pen employed in the previously disclosed example embodiments shown in the several drawing figures discussed herein above. Tool 800 comprises a base 802 fit with a generally cylindrical pen holder 804 for fixturing injection pen 10. Base 802 is fit with a guide slot 806. A blade guide 807 contains the parting mechanism. Blade guide 807 comprises a guide block 808 which is keyed to fit guide slot 806. Similarly, guide block 808 is fit with a complementary slot to accommodate a cutting blade 810 which is held in place with a retaining pin 812.

Pen 10 is fixtured by inserting it into holder 804. Blade guide 807 is then inserted into guide slot 806. The complementary shapes of pen holder 804, guide slot 806 and guide block 808, as shown in FIGS. 13A-B, for example, are configured such that blade 810 is centered over the long axis of the pen injector 10. The parting action of blade 810 is accomplished by sliding blade guide 808 in the direction indicated by the linear arrow shown in FIG. 13A. During the sliding action, a spring 814 in compression urges blade 810 into contact under force with cartridge holder 14 as the contour of cartridge holder 14 changes. Blade guide 808 may be returned to its starting position and the sliding action may be repeated until cartridge holder 14 is parted, in some embodiments. Pen injector 10 is then removed, and the glass and plastic materials of cartridge 14 are separated for recycling and disposal.

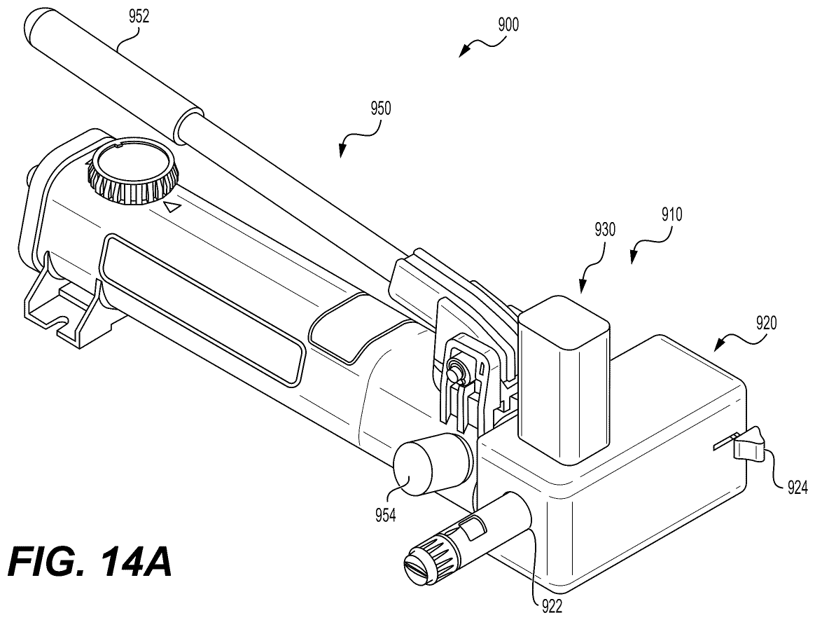

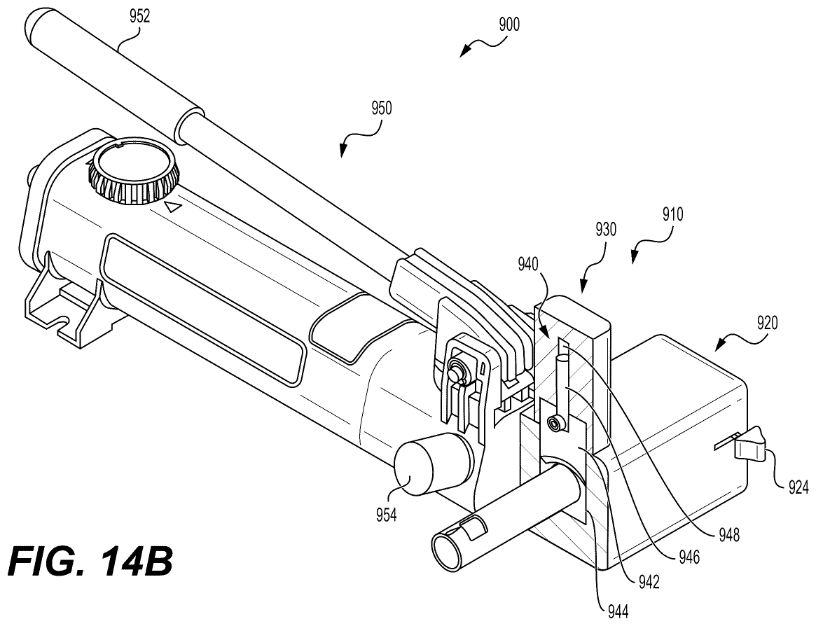

FIGS. 14A-B show an additional example embodiment of an injector disassembly tool 900. Disassembly tool 900 comprises a housing 910 having a fixing section 920 and a parting section 930. Fixing section 920 includes an injector holder 922 which is generally formed as a hollow cylinder, in some embodiments, which receives a distal end of pen injector 10. A distal end of holder 922 includes a stop block (not shown) which can be moved axially (with respect to pen 10) by the user via a depth selector 924. This controls the depth of insertion of the pen injector 10 into channel 944. Parting section 930 includes a guillotine-style cutter 940. Cutter 940 comprises a cutting blade 942 constrained within housing 910 by one or more vertical channels 944 configured to allow blade 942 to freely move vertically up and down. Blade 942 is coupled to a piston 946 fit within a bore 948 so as to freely travel up and down.

Cutter 940 is actuated in this example and in some embodiments via a hydraulic or pneumatic hand pump 950, similar to those pneumatic hand pumps of which the design and operation are known in the art. By the user grasping and pumping a handle 952, hydraulic fluid is moved under pressure into the bore 948, forcing piston 946 and blade 942 downward. The downward force of blade 942 shears a housing of pen injector 10 just proximal to the glass cartridge. The user then retracts blade 942 by opening a release valve 954, thereby releasing the pressure of the hydraulic fluid within bore 948. Moving depth selector 924 in a proximal direction following the parting action and retraction of blade 942 allows the parted pieces of pen injector 10 to be removed from injector disassembly tool 900 followed by separation of the glass cartridge from the remaining plastic for separate recycling and disposal.

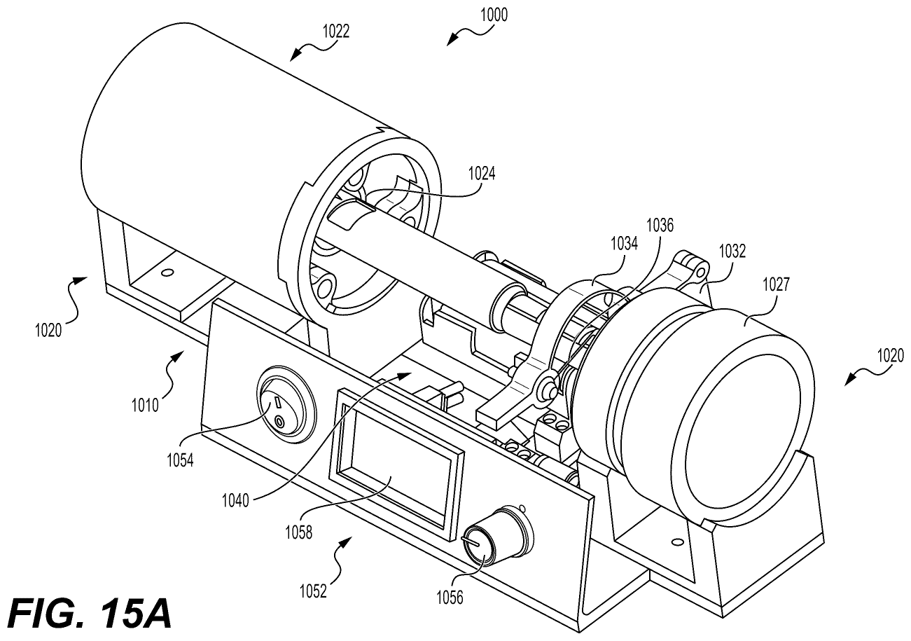

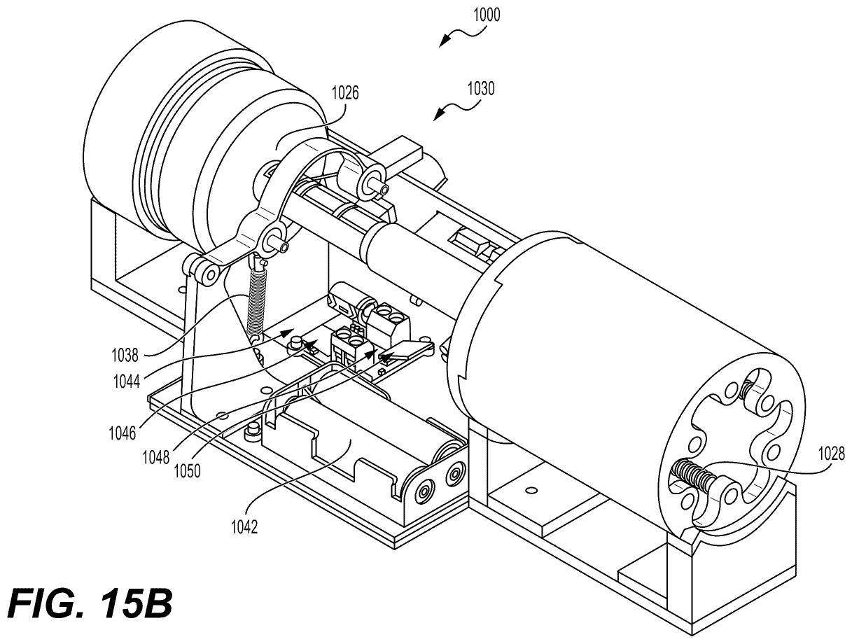

FIGS. 15A-B show an additional example embodiment of an injector disassembly tool 1000. Tool 1000 comprises a housing 1010 containing a fixing section 1020, a parting section 1030, and a power and control section 1040. A live center 1022 features a hollow cone 1024 which accommodates the proximal end of pen injector 10 and keeps injector 10 axially centered. Cone 1024 is mounted to a carriage which translates axially. The distal end of pen injector 10 is coupled to a drive coupler 1026 by interaction of complementary male and female threads, in some embodiments. One or more compression springs 1028 attached to the carriage of cone 1024 restrict axial movement of cone 1024 when pen injector 10 is coupled to coupler 1026, wherein injector disassembly tool 1000 may accommodate various lengths of pen injector 10 and various diameters of dosing knob 18. Coupler 1026 is coupled to an output shaft of a motor 1027.

The parting mechanism in some embodiments, such as the embodiment shown in FIGS. 15A-B, for example, includes a “hot knife” cutter for structures formed from plastic materials, the design and operation of which is known in the art. Parting section 1030 comprises a lever arm 1034 mechanically coupled to a pivot disposed on a first end of a pylon 1032. In some embodiments, a heating element 1036 is fixed to lever arm 1034 and held in contact with cartridge holder 14 of pen injector 10 by a tension spring 1038. In some embodiments, heating clement 1036 comprises a nichrome wire.

A power and control section 1040 includes a power supply 1042. In some embodiments, power supply 1042 is a transformer circuit configured for an external power supply, or a self-contained internal power supply such as a rechargeable battery, for example. Power supply 1042 supplies power to a motor 1027, heating element 1036, a control board 1044 bearing control electronics 1046, a microprocessor 1048, and a memory 1050 wherein a controlling software resides. A front panel 1052 includes an on/off power switch 1054, a heating element temperature controller 1056 and a display 1058.

In a configuration whereunder pen injector 10 is coupled to disassembly tool 1000, a desired heating element temperature can be set, and power switched on by activating switch 1054. Motor1028 is activated by the controlling software causing drive coupler 1026 to rotate the coupled pen injector 10 to axially rotate. Heating element 1036 is energized by the controlling software providing power to element 1036 and brought to a target temperature sufficient to melt or otherwise soften the plastic of injection pen cartridge holder 14 sufficient to fully or partially part cartridge holder 14. After terminating power to injector disassembly tool 1000 and allowing time for heating element 1036 to sufficiently cool such that pen injector 10 can be safely handled, the parted pen injector 10 pieces may be removed from tool 1000 and the glass cartridge separated from the remaining plastic for separate recycling and disposal.

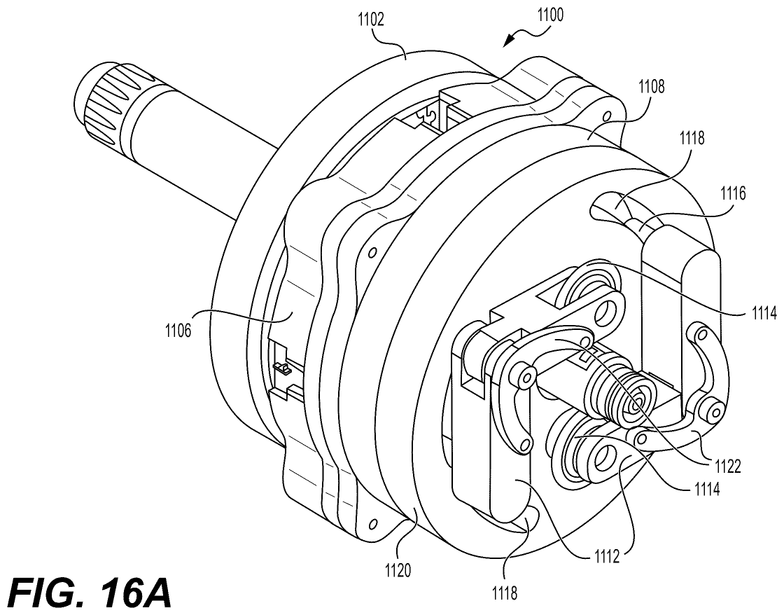

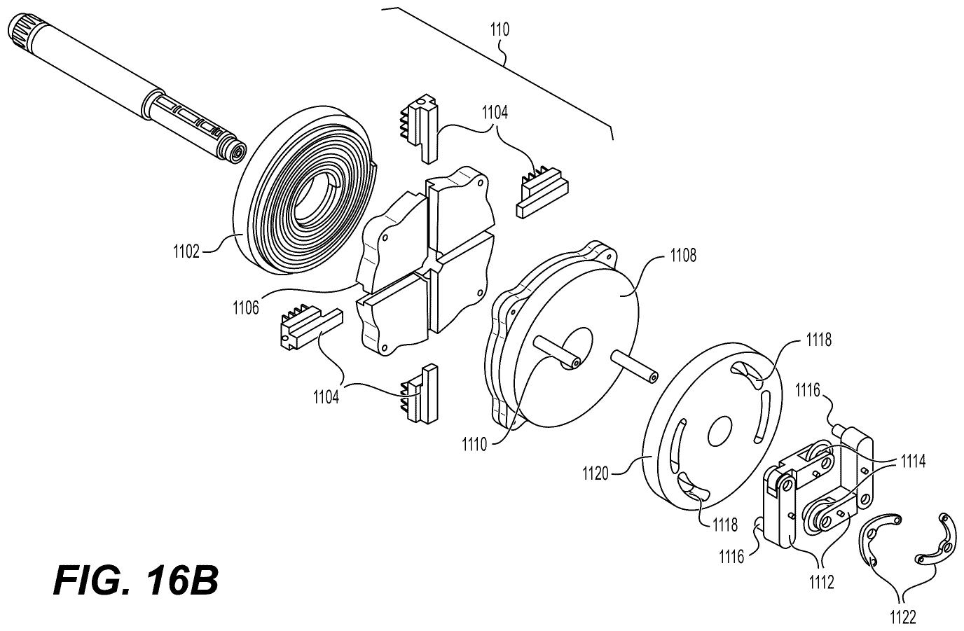

There are additional alternative mechanisms for coupling and parting pen injector 10 in an injector disassembly tool. FIGS. 16A-B show an example embodiment of an injector disassembly tool 1100 comprising a four-jaw self-centering scroll chuck mechanism for coupling to pen injector 10. Rotating a scroll plate 1102 closes and opens jaws 1104 mounted within a chuck body 1106. Pen injector 10 is inserted through chuck 1100 and fixed in placed by closing jaws 1104, clamping injector pen 10 in place.

FIGS. 16A-B also show injector disassembly tool 1100 comprising an alternative parting mechanism. A base plate 1108 is coupled to chuck body 1106 such that base plate 1108 is free to axially rotate. One or more posts 1110 create a pivot point of pivot arms 1112. The pivot point of pivot arms 1112 is hinged. A first end of each pivot arm 1112 holds one or more cutting wheels 1114. A cam follower 1116 is coupled to a second end of each pivot arm 1112. Cam followers 1116 are configured to interact with a corresponding one or more slots 1118 within a cam plate 1120. Rotation of cam plate 1120 causes pivot arms 1112 to pivot whereunder a distance between cutter wheels 1114 is varied.

The example embodiment of tool 1100 shown in FIGS. 16A-B comprises an alternative mechanism for limiting an applied force that cutting wheels 1114 exert against coupled pen injector 10 so as not to break glass cartridge 16 during the parting action. As cam plate 1120 is axially rotated relative to base plate 1108, cutting wheels 1114 are brought into contact with the cartridge holder 14 of pen injector 10, and pivot arms 1112 begin to hinge open. One or more live springs 1122 straddle each corresponding pivot arm applying a controlled resisting force. During the parting process, base plate 1108 and cam plate 1120 rotate together relative to chuck 1100 wherein cutter wheels 1114 exert a parting force on cartridge holder 14. Live springs 1122 impart a varying force to each cutter wheel 1114 as the distance between the wheels varies with the rotation around the cartridge holder sufficient to maintain a cutting and parting action. Once cartridge holder 14, cam plates 1120 are rotated in the opposite axial direction such that pivot arms 1112 reverse pivot causing cutter wheels 1114 to retract. Live springs 1122 then return to an initial baseline starting. position.

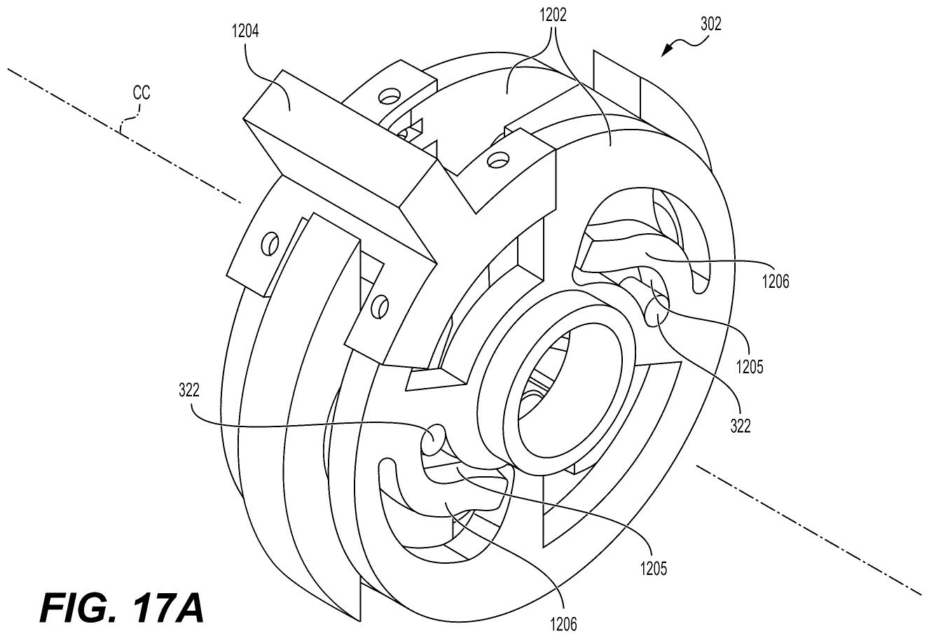

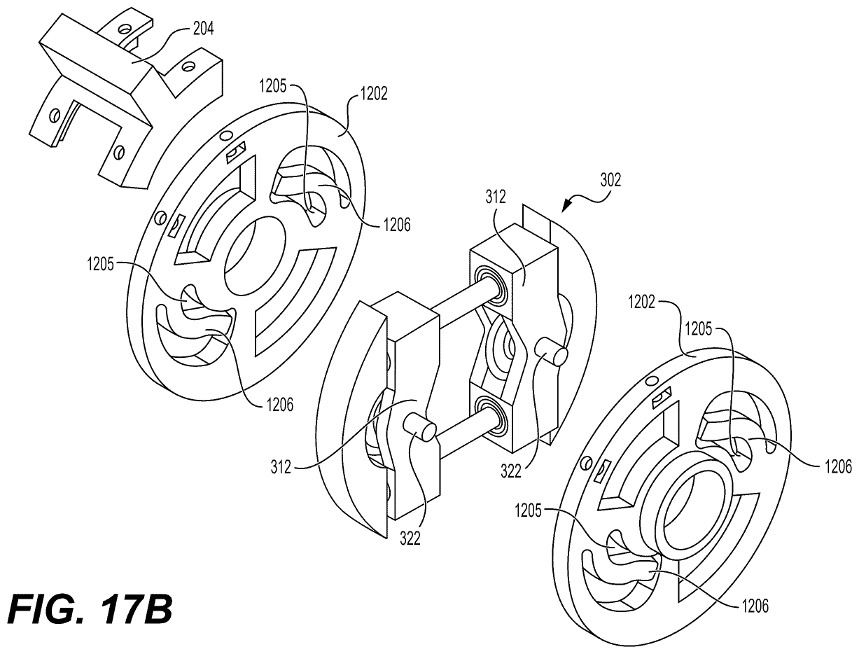

FIGS. 17A-B show an alternative mechanism for actuating the parting mechanism and limiting the applied force of a cutter wheel cartridge 302 on cartridge holder 14. One or more cutter cams 1202 each include a slot 1205 having a deformable wall 1206. Each slot 1205 receives a corresponding axle shaft protrusion 322. A profile of each slot 1206 is configured wherein by rotating cutter cams 1202 about an axis CC, a distance between each cutter wheel carrier 312 and its cutter wheel 310 can be changed. A lever 1204 couples two cams 1202. With pen injector 10 coupled to an injector disassembly tool along axis CC in an injector disassembly tool having cutter cam 1202, rotating cam(s) 1202 by actuating lever 1204 urges cutter wheels 310 into engagement with cartridge holder 14. Deformable wall 1206 acts as a live hinge and controls the magnitude of a force applied by cutting wheel(s) 1114 on cartridge holder 14. After cartridge holder 14 is parted and each cam 1202 is axially rotated in the opposite direction, each cutter wheel carrier 312 retracts and each deformable well 1206 returns to a baseline resting position.

There are various possibilities for implementing the housing, fixing, and parting mechanism of different embodiments of a pen injector disassembly tool. In addition to those described in the foregoing examples, a variety of parting tools are envisioned. Different shaped cutting blades may have advantages in terms of manufacturing cost, longevity, or ease of use.

It is anticipated that manufacturers of pen injectors and similar injector devices, in response to demand for less single-use plastic in their injection pen offerings, may begin to introduce different materials for forming cartridge holders. Such materials may include metal such as aluminum. Materials may also include wood, bamboo, bagasse, or composites. Cutter wheels may include serrated, abrasive, or other mechanical cutting edge configurations to accommodate injector bodies and cartridge holders made from materials other than plastic. Heat or vibration, whether low-frequency, high-frequency, or ultrasonic frequency, may also facilitate easier parting of injector bodies regardless of the material used in its manufacture.

While the example embodiments above include injector fixing and parting elements and mechanisms that allow for different profiles and lengths, it is envisioned that an injector disassembly tool could include fixing mechanisms or parting mechanisms having a modular design specific to the design of a particular manufacturer's injector. The modular design enables a user of the injector disassembly tool to simply exchange fixing and parting modules particular to a given manufacturer. In addition, such a modular design would facilitate replacement of worn or broken disassembly tool elements to simplify any necessary maintenance or repair of the disassembly tool.

It is additionally envisioned that the pen recycler can be designed and manufactured for use with a particular manufacturer's pen design. This would simplify the fixing mechanism and the parting mechanism of the injector disassembly tool, wherein the size, number of individual parts, and usage steps are reduced, thus improving usability. Further, these simplifications could reduce or eliminate the need for disassembly tool replacement when parts of a single module become worn or damaged.

A variety of construction materials for an injector disassembly tool may be used, including various types of plastic in some embodiments. Given the aim of an injector disassembly tool is to reduce the amount of single-use plastic in pen injectors, constructing a disassembly tool from environmentally sensitive materials would be beneficial. Non-limiting examples of such materials include recyclable metal, wood, and other fibers such as bagasse. Recycled plastic, bio plastics, and compostable bioplastics are also alternatives.

Some embodiments of an injector disassembly tool may incorporate a means for disassembling a pen injector cartridge. For example, a glass cartridge may be isolated by decrimping crimp cap 34 and septum 32 and dislodging piston 28. Apparatus and methods for decrimping vials and cartridges of liquid medicaments and perfumes are known in the art. Piston 28 may be easily removed by injecting air or another fluid under pressure through septum 32 before decrimping or by a mechanical means after crimp cap 34 and septum are removed. Glass cartridge 16 could then be rinsed, cleaned, disinfected, and recycled by the user.

Any injector disassembly tool that only partially disaggregates a pen injector may have user advantages. If the user is able to twist or pivot the injection pen about the partial parting such that a glass cartridge can be removed without fully parting the pen body, fewer pieces will be created for recycling. It also may make uncoupling and removal of the injector from the disassembly tool following operation more efficient. Fixing mechanisms that limit the extent of the parting action or parting mechanisms that limit depth of cut of the injector body or provide intermittent cutting, such as a perforation, may be included in a partial disassembly mechanism.

Electromechanical systems for automating operations of an injector disassembly tool are used in some embodiments. Such systems may be especially useful in institutional and clinical environments with a need to disassemble high volumes of pen injectors for recycling. Also, electromechanical systems may have increased utility for users with diminished dexterity, strength, or vision. Such electromechanical systems incorporate motors, actuators, electronics, and control software to the mechanisms described herein.

Several embodiments of an injector disassembly tool and methods of use have been described herein. The injector disassembly tool provides a means for separation of component parts of a patient-use injector comprising different materials, such as a pen injector having a plastic cartridge holder bearing a glass medicament cartridge, for separate recycling of plastic, glass, and other materials. Recycling of pen injectors and related limited-use injector devices reduces waste and increases sustainability of manufacture in an environmentally sensitive manner. This is particularly important in today's market wherein the demand for manufacturing and subsequently disposing of limited-use personal medicament injector devices is anticipated to continue increasing.

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purpose of illustration and example. The description as set forth is not intended to be exhaustive or to limit the invention to the precise form disclosed. Many modifications and variations are possible, in light of the teachings herein above.

We claim:

1. A multidose pen injector disassembly tool comprising:
a holder section having a fixing means configured to couple to a disposable injector;
a parting mechanism having a cutter, wherein the parting mechanism is configured to divide and separate an injection pen housing from a drug cartridge contained within the injection pen housing;
a drive section mechanically coupled to the parting mechanism comprising a torque-limiting driver such that the drug cartridge can be removed from the injection pen housing without breaking the drug cartridge and
a coupler section;
wherein the holder section, the parting mechanism, drive section, and the coupler section are sequentially coupled end-to-end along a central longitudinal axis.

2. The multidose pen injector disassembly tool of claim 1, wherein the fixing means comprises a female threaded fixture.

3. The multidose pen injector disassembly tool of claim 1, wherein the holder section, the parting mechanism, the drive section, and the coupler section are reversibly coupled through an interaction of interlocking surface features joining a housing of each section.

4. The multidose pen injector disassembly tool of claim 1, wherein the parting mechanism comprises a cutter wheel.

5. The multidose pen injector disassembly tool of claim 1, wherein the parting mechanism comprises a cutting wire.

6. The multidose pen injector disassembly tool of claim 1, wherein the parting mechanism comprises a cutting blade.

7. The multidose pen injector disassembly tool of claim 1, wherein the cutter section comprises:
a cutter wheel cartridge configured to insert into a cutter housing, the cutter wheel cartridge having
a frame coupled to a pair of parallel guide rails,
a pair of diametrically opposed cutter wheel carriers fixed in a plane perpendicular to the central longitudinal axis by the pair of parallel guide rails and fitted with a linear bearing that rides on the pair of parallel guide rails configured such that each carrier is free to move independently along the guide rails; and
a cutter wheel held within each cutter wheel carrier.

8. The multidose pen injector disassembly tool of claim 7 wherein the cutter section comprises a cutter cam, wherein each cutter wheel is mounted on an axle shaft, wherein the axle shaft includes a shaft protrusion received by a curved slot in the cutter cam configured such that by rotating the cutter cam about the central longitudinal axis, the cutter wheel is urged into engagement with an outer surface of the pen injector fixed within the injector disassembly tool.

9. The multidose pen injector disassembly tool of claim 8, wherein the drive section further comprises a torque limiting driver assembly configured to limit a torque force applied by the cutter wheel to the outer surface of the pen injector.

10. A multidose pen injector disassembly tool comprising:
a holder section having a base having a fixing means configured to couple to a disposable pen injector;
a parting mechanism having a cutter;
a drive section mechanically coupled to the cutter section and
a coupler section;
wherein the holder section, the parting mechanism, the drive section, and the coupler section are sequentially coupled end-to-end along a central longitudinal axis, and
wherein the injector disassembly tool is configured to divide a housing section of the pen injector coupled to the base by rotating the cutting section about the central longitudinal axis relative to the base.

11. The multidose pen injector disassembly tool of claim 10, wherein the cutter section comprises a cutter wheel.

12. The multidose pen injector disassembly tool of claim 10, wherein the cutter section comprises a cutting wire.

13. The multidose pen injector disassembly tool of claim 10, wherein the cutter section comprises a cutting blade.

14. The multidose pen injector disassembly tool of claim 10, wherein the cutter section comprises a hot knife.

15. The multidose pen injector disassembly tool of claim 10, wherein the parting mechanism is configured to receive a shape of at least one commercially available pen injector.

16. The multidose pen injector disassembly tool of claim 10 having a generally uniform cylindrical shape.

17. A method of use for a multidose pen injector disassembly tool comprising:
an inserting step threading a pen injector comprising a housing and a drug cartridge therein into a threaded fixture on a base of an injector disassembly tool;
a coupling step stabilizing the pen injector with a live center mechanism;
a rotating step manually rotating a handle to engage a clutch mechanisms mechanically coupled to a cutter wheel configured to part the housing without breaking the drug cartridge;
a cutting step cutting the housing with the cutter wheel such that the drug cartridge can be removed from the housing without breaking the drug cartridge;
a releasing step releasing the pen injector from the injector disassembly tool; and
a decoupling step removing the drug cartridge from the housing.

18. The method of claim 17, wherein the inserting step comprises threading a multidose pen injector into a threaded fixture on a base of an injector disassembly tool.

19. The method of claim 17, wherein the coupling step comprises stabilizing the pen injector in a position along a central longitudinal axis with a live center mechanism configured to accommodate a range of pen injector lengths and diameters.

20. The method of claim 17, wherein the rotating step comprises manually rotating a handle to engage a clutch mechanism mechanically coupled to a cutter wheel.
